(19) United States
(12) Patent Application Publication
(10) Pub. No.: US 2022/0230753 A1
(43) Pub. Date: Jul. 21, 2022

(54) TECHNIQUES FOR EXECUTING TRANSIENT CARE PLANS VIA AN INPUT/OUTPUT DEVICE

(71) Applicant: Intuition Robotics, Ltd, Ramat-Gan (IL)

(72) Inventors: Shay ZWEIG, Harel (IL); Alex KEAGEL, Tel Aviv (IL); Chen SORIAS, Kibbutz Zikim (IL); Dor SKULER, Oranit (IL)

(73) Assignee: Intuition Robotics, Ltd., Ramat-Gan (IL)

(21) Appl. No.: 17/647,771

(22) Filed: Jan. 12, 2022

Related U.S. Application Data
(60) Provisional application No. 63/138,671, filed on Jan. 18, 2021.

(51) Int. Cl. G16H 50/20

(52) U.S. Cl. CPC G16H 50/20; G06Q 10/1093

(57) ABSTRACT

A system and method for executing transient care plans for a user via an input/output device is provided. The method includes determining at least one transient care plan based on a care plan of a user from a medical entity, a set of predefined health-related guidelines, and at least one user dataset captured by an input/output (I/O) device, wherein a transient care plan is a customized care plan for the user; creating, based on the at least one transient care plan and the at least one user dataset, an estimated schedule, wherein the estimated schedule includes a plurality of rules for executing a portion of the at least one transient care plan; and projecting at least one first plan via the I/O device, wherein the at least one first plan is identified from the at least one transient care plan based on the estimated schedule.

### CROSS-REFERENCE TO RELATED APPLICATIONS

This application claims the benefit of U.S. Provisional Application No. 63/138,671 filed on Jan. 18, 2021, the contents of which are hereby incorporated by reference.

### TECHNICAL FIELD

The disclosure generally relates to digital assistants operated in an input/output (I/O) device, and more specifically to techniques for executing transient care plans for a user of I/O devices.

### BACKGROUND

As manufacturers improve the functionality of devices such as vehicles, computers, mobile phones, appliances, and the like, through the addition of digital features, manufacturers and end-users may desire enhanced device functionalities. The manufacturers, as well as the relevant end-users, may desire digital features which improve user experiences, interactions, and features which provide for greater connectivity. Certain manufacturers may include device-specific features, such as setup wizards and virtual assistants, to improve device utility and functionality. Further, certain software packages may be added to devices, either at the point of manufacture, or by a user after purchase, to improve device functionality. Such software packages may provide functionalities including, as examples, a computer system's voice control, facial recognition, biometric authentication, and the like.

While the features and functionalities described hereinabove provide for certain enhancements to a user's experience when interacting with a device, the same features and functionalities, as may be added to a device by a user or manufacturer, fail to include certain aspects which may allow for a further-enhanced user experience. First, certain currently-implemented digital assistants and other user experience features may fail to provide for adaptive adjustment of the operation of the assistant or feature. For example, a digital assistant configured to play music may be programmed to use a specific type of music streaming services, thereby limiting the user experience. In addition, certain currently-implemented digital assistants and other user experience features may fail to provide for adjustments of the assistant or feature operation based on context or environmental data. As an example, a digital assistant may be configured to present reminders to take vitamins at a certain time. However, such reminder may be inappropriate and disturbing when the user is surrounded by guests or at certain hours of the day.

Such adaptive adjustment of operation of the assistant or feature may be particularly important and useful for assisting users in health-related operations that are closely related to the well-being of a user. As an example, when there are two users in the same room with different health conditions, a suggestion regarding one health condition must be direct to the correct user. Similarly, a workout to satisfy the health-related operation of exercising 20 minutes per day, for example, should be different for a marathon runner and a patient recovering from surgery. Furthermore, an appropriate time and means of presenting such suggestions is important for such personal and private information. However, the currently-implemented digital assistants often lack such adaptiveness for each of the multiple users and their needs. As such, users may not benefit from policies or actions performed or suggested by the digital assistants, and eventually abandon the usage of such device.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for executing transient care plans for a user via an input/output device. The method comprises: determining at least one transient care plan based on a care plan of a user received from a medical entity, a set of predefined health-related guidelines, and at least one user dataset captured by an input/output (I/O) device, wherein a transient care plan is a customized care plan for the user; creating, based on the at least one transient care plan and the at least one user dataset, an estimated schedule, wherein the estimated schedule includes a plurality of rules for executing a portion of the at least one transient care plan; and projecting at least one first plan via the I/O device, wherein the at least one first plan is identified from the at least one transient care plan based on the estimated schedule.

Certain embodiments disclosed herein also include a non-transitory computer readable medium having stored thereon causing a processing circuitry to execute a process, the process comprising: determining at least one transient care plan based on a care plan of a user received from a medical entity, a set of predefined health-related guidelines, and at least one user dataset captured by an input/output (I/O) device, wherein a transient care plan is a customized care plan for the user; creating, based on the at least one transient care plan and the at least one user dataset, an estimated schedule, wherein the estimated schedule includes a plurality of rules for executing a portion of the at least one transient care plan; and projecting at least one first plan via the I/O device, wherein the at least one first plan is identified from the at least one transient care plan based on the estimated schedule.

Certain embodiments disclosed herein also include a system for executing transient care plans for a user via an input/output device. The system comprises: a processing circuitry; and a memory, the memory containing instructions that, when executed by the processing circuitry, configure the system to: determine at least one transient care plan based on a care plan of a user received from a medical entity, a set of predefined health-related guidelines, and at least one user dataset captured by an input/output (I/O) device, wherein a transient care plan is a customized care plan for the user; create, based on the at least one transient care plan and the at least one user dataset, an estimated schedule, wherein the estimated schedule includes a plurality of rules for executing a portion of the at least one transient care plan; and project at least one first plan via the I/O device, wherein the at least one first plan is identified from the at least one transient care plan based on the estimated schedule.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe the various embodiments of the disclosure.

FIG. 2 is a block diagram of a controller, according to an embodiment.

FIG. 3 is a flowchart illustrating a method for determining transient care plans and an estimated schedule for execution of plan for a user of the digital assistant according to an embodiment.

FIG. 4 is a flowchart illustrating a method for updating an estimated schedule for a user of a digital assistant according to an embodiment.

FIG. 5 is a flowchart illustrating a method for modifying an estimated schedule based on a current state of a user of the digital assistant according to an embodiment.

### DETAILED DESCRIPTION

The embodiments disclosed by the disclosure are only examples of the many possible advantageous uses and implementations of the innovative teachings presented herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality. In the drawings, like numerals refer to like parts through several views.

The various embodiments disclosed herein provide techniques for effectively and accurately executing transient care plans for a user of the digital assistant by incorporating a dataset of the user. The transient care plans may be customized health regiments that are determined for each of the multiple users of the digital assistant based on a care plan from a medical entity, health-related guidelines of the digital assistant, and the dataset of each user that is captured by the I/O device operating the digital assistant. The transient care plans are determined to accurately represent and adhere to the care plans from the medical entity, yet tailored to the condition (e.g., preference, pattern, habit, physical and emotional state, and so on) of the user of the digital assistant. Moreover, an estimated schedule including a plurality of execution rules is created for at least a portion of the determined transient care plan with respect to the dataset of the user. That is, a suggestion or reminder for a user to perform a transient care plan may be projected at certain times through the I/O device according to a user preference, behavior, and the like. Furthermore, the personalized transient care plan and the estimated schedule may be updated and/or modified to more accurately serve each user in different circumstances based on analyses of the sensory data, as may relate to a user, the conditions of the user's environment, and the like.

It should be appreciated that presenting the transient care plan at appropriate and desirable timings, with respect to time of day, weather, condition of user, and the like, for each of the user may be critical in actual execution of the health-related care plan by the user. For certain health-related care plans, actual conduction of the care plan may be directly related to the user's health condition and well-being and thus, increasing compliance with the care plan is highly desired. To this end, in an embodiment, the estimated schedule including projection rules for the transient care plan are created to improve compliance and user performance of the care plan suggested by the medical entity. Moreover, user's current state and/or feedback data may be determined to further update the estimated schedule for improved accuracy.

In an embodiment, the current state and the feedback data may be determined based on real-time data of the user and the user's environment and user responses as collected by the I/O device near (e.g., in a predetermined proximity to) the user. The disclosed embodiments enable immediate and continuous monitoring of the user's condition and compliance with the health-related care plan and dynamic updating of the rules of the estimated schedule. That is, the transient care plan and the projection according to the estimated schedule may better reflect the needs and preference of each user of the digital assistant to increase compliance of the user to their much-needed execution of the health-related activity (i.e., plan).

The embodiments disclosed herein provide advantageous objective rules-based analyses of the dataset of the user collected by the I/O devise to increase consistency and accuracy in interacting with the user of the digital assistant in regard to health-related care plans. While individuals (e.g., spouse, doctor, etc.) may present and record user's response to care plans, decisions such as, but not limited to, what and when to project, as well as the analyses of sensor data can be highly subjective. In this approach, each individual that participates may subjectively determine the plan and schedule to project the plan based on their “feeling” of the user's current state and response to previously presented care plans, where the same user response may provide a different “feeling” for each individual to result inconsistent decision and output.

However, a digital assistant, as disclosed, provides rules defined by, for example, weights, scores, ranking, and the like of certain parameters, to objectively analyze the dataset of the user. In an embodiment, such objectively defined parameters are utilized to further modify and improve the transient care plan and the estimated schedule to appropriately present the health-related care plan to each user. Moreover, the digital assistant of the embodiment, allows immediate tracking of the user's condition and response to increase the accuracy of the transient care plan itself as well as the timing, which is otherwise difficult for individuals (e.g., spouse, doctor, etc.) to track and perform.

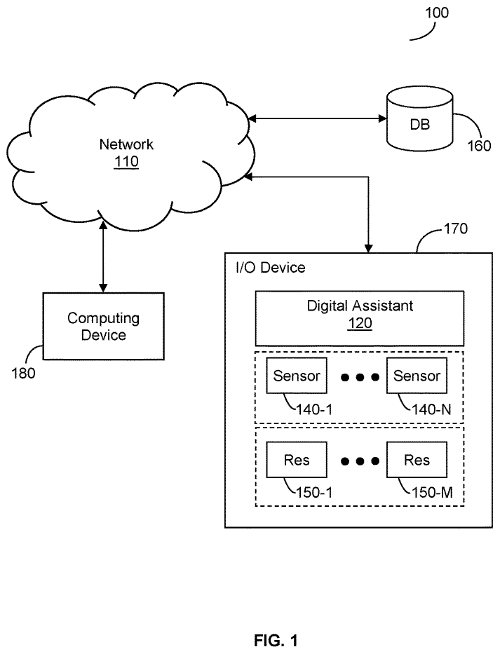

FIG. 1 is an example network diagram 100 utilized to describe the various disclosed embodiments. The network diagram 100 includes an input/output (I/O) device 170 operating a digital assistant 120. In some embodiments, the digital assistant 120 is further connected to a network 110 to allow some processing of a remote server (e.g., a cloud server). The network 110 may provide for communication between the elements shown in the network diagram 100. The network 110 may be, but is not limited to, a local area network (LAN), a wide area network (WAN), a metro area network (MAN), the Internet, a wireless, cellular, or wired network, and the like, and any combination thereof.

In an embodiment, the digital assistant 120 may be connected to, or implemented on, the I/O device 170. The I/O device 170 may be, for example and without limitation, a robot, a social robot, a service robot, a smart TV, a smartphone, a wearable device, a vehicle, a computer, a smart appliance, and the like.

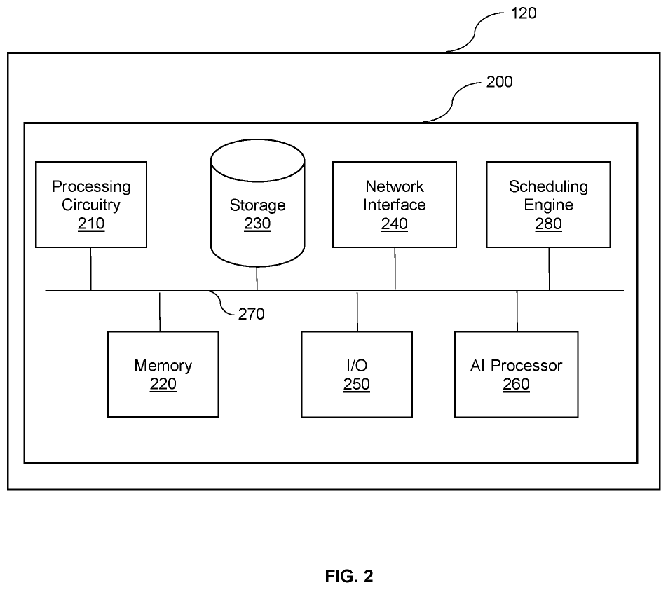

The digital assistant 120 may be realized in software, firmware, hardware, and any combination thereof. An example block diagram of a controller that may execute the processes of the digital assistant 120 is provided in FIG. 2. The digital assistant 120 is configured to process sensor data collected by one or more sensors, 140-1 to 140-N, where N is an integer equal to or greater than 1 (hereinafter referred to as “sensor” 140 or “sensors” 140 for simplicity) and one or more resources 150-1 to 150-M, where M is an integer equal to or greater than 1 (hereinafter referred to as “resource” 150 or “resources” 150 for simplicity). The resources 150 may include, for example, electro-mechanical elements, display units, speakers, and the like. In an embodiment, the resources 150 may include sensors 140 as well. The sensors 140 and the resources 150 are included in the I/O device 170.

The sensors 140 may include input devices, such as various sensors, detectors, microphones, touch sensors, movement detectors, cameras, and the like. Any of the sensors 140 may be, but are not necessarily, communicatively, or otherwise connected to the digital assistant 120 (such connection is not illustrated in FIG. 1 for the sake of simplicity and without limitation on the disclosed embodiments). The sensors 140 may be configured to sense signals received from a user interacting with the I/O device 170 or the digital assistant 120, signals received from the environment surrounding the user, and the like. In an embodiment, the sensors 140 may be implemented as virtual sensors that receive inputs from online services, for example, the weather forecast, a user's calendar, and the like.

In an embodiment, a database (DB) 160 may be utilized. The database 160 may be part of the I/O device 170 (e.g., within a storage device not shown), or may be separate from the I/O device 170 and connected thereto via the network 110. The database 160 may be utilized for storing, for example, health-related information of one or more users of the digital assistant, predefined health-related guidelines of the digital assistant, historical information of the user (e.g., patterns, preferences, and so on), and the like, as well as any combination thereof.

In an embodiment, a computing device 180 may be communicatively connected to the I/O device 170 via the network 110. The computing device 180 may be a personal computer (PC), a laptop, a server, and the like that is associated with an entity such as, but not limited to, a medical entity. A medical entity may be for example, a medical team, a doctor, and the like. The computing device 180 may be configured to communicate with the digital assistant 120 through the network 110.

FIG. 2 is an example block diagram of a controller 200 acting as a hardware layer of a digital assistant 120, according to an embodiment. The controller 200 includes a processing circuitry 210 that is configured to receive data, analyze data, generate outputs, and the like, as further described hereinbelow. The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 200 further includes a memory 220. The memory 220 may contain therein instructions that, when executed by the processing circuitry 210, can cause the controller 200 to execute actions as further described hereinbelow. The memory 220 may further store therein information, for example but not limited to, data associated with one or more users, health-related information of one or more users, predetermined health parameters, values associated with the health parameters, historical information of the user (e.g., patterns, preferences, etc.), various feedback data (e.g., a user response, a user reaction, etc.), and the like.

The storage 230 may be magnetic storage, optical storage, and the like, and may be realized, for example, as a flash memory or other memory technology, or any other medium which can be used to store the desired information.

In an embodiment, the controller 200 includes a network interface 240 that is configured to connect to a network, e.g., the network 110 of FIG. 1. The network interface 240 may include, but is not limited to, a wired interface (e.g., an Ethernet port) or a wireless port (e.g., an 802.11 compliant Wi-Fi card), configured to connect to a network (not shown).

The controller 200 further includes an input/output (I/O) interface 250 configured to control the resources (e.g., 150, FIG. 1) which are connected to the digital assistant 120. In an embodiment, the I/O interface 250 is configured to receive one or more signals captured by the sensors (e.g., 140, FIG. 1) of the digital assistant (e.g., 120, FIG. 1) and to send such signals to the processing circuitry 210 for analysis. In an embodiment, the I/O interface 250 is configured to analyze the signals captured by the sensors 140, detectors, and the like. In a further embodiment, the I/O interface 250 is configured to send one or more commands to one or more of the resources 150 for executing one or more plans (e.g., actions) of the digital assistant 120, as further discussed herein below. A plan may include, for example, presenting a portion of the determined transient care plan for the user based on an estimated schedule created according to an embodiment. As an example, a plan may include suggesting the user to timely take her or his medications, play a cognitive game, suggesting the user to go out for a walk, reminding the user to go to yoga class, and so on. In further embodiment, the components of the controller 200 are connected via a bus 270. A care plan is a health regimen that is uniquely determined for a person such as the user of the digital assistant 120. The transient care plan, such as a health-regiment, may be determined based on the unique care plan for further personalization for each of the users of the digital assistant 120.

In some configurations, the controller 200 may further include an artificial intelligence (AI) processor 260. The AI processor 260 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI processor 260 is configured to perform, for example, machine learning based on sensory inputs received from the I/O interface 250, where the I/O interface 250 receives input data, such as sensory inputs, from the sensors 140.

In an embodiment, the controller 200 may further include a scheduling engine 280. The scheduling engine 280 is configured to receive and analyze several datasets (such as a transient care plan, predefined health-related guidelines of the digital assistant 120 and dataset gathered with respect to the user) in order to determine and generate an estimated schedule for projecting at least a portion of a transient care plan. A transient care plan is a health regimen that is customized to the user based on, among other things, the user's behavior, patterns, habits, preferences, and the like as further discussed herein below. In an embodiment, the scheduling engine 280 may further be configured to present or communicate the estimated schedule to the user, monitor the user response with respect to the estimated schedule, adjust the estimated schedule, and the like.

In an embodiment, the controller 200 may receive from a computing device (e.g., the computing device 180, FIG. 1) that is associated with a medical entity, a care plan that is related to the user. As noted above, a care plan is a health regimen that is uniquely determined for a person such as the user of the digital assistant 120. The care plan may include medical instructions such as, take a certain medication at a specific timing, do physical activity three times a week, avoid red meat, and so on. In an embodiment, the care plan of the user may be sent from the computing device 180 through the network 110 to the digital assistant 120 using a web interface. According to a further embodiment, the care plan of the user may be fed into an electronic medical record (EMR) of the user and thus, the digital assistant 120 may be configured to collect the care plan (or updates related to the care plan) from the EMR of the user.

In an embodiment, the controller 200, when executing the digital assistant 120, may be configured to analyze the care plan with at least a dataset of the user and a set of predefined health-related guidelines of the digital assistant 120. A dataset of the user may include at least historical data of the user (such as, preferences, behavioral patterns, and so on). In an embodiment, the dataset may also include real-time data that is being collected in real-time with respect to the user and the user's environment.

The dataset of the user may be collected through time using sensors (e.g., the sensors 140) that are communicatively connected to and controlled by the digital assistant 120. That is, the dataset may include sensory inputs that are analyzed using, for example, machine learning algorithm that is adapted to interpret the meaning of certain scenarios (e.g., user is alone, has company, asleep, angry, sad, happy, watching TV, and the like). By collecting and analyzing sensory inputs that may be stored in a database (e.g., the database 160, FIG. 1), user preferences, habits, behavioral patterns, and the like may be determined.

The set of predefined health-related guidelines of the digital assistant 120 may refer to predetermined rules. Such health-related rules or guidelines may indicate for example that: the user should drink 6-8 glasses of water per day, the user should sleep at least 7 hours at night, the user should not be inactive for more than four hours in a row during daytime, and so on. The analysis of the care plan with the dataset of the user and the set of predefined health-related guidelines may be achieved by applying at least one algorithm, such as a machine learning algorithm, that is adapted to determine a transient care plan. A transient care plan may refer to a health regimen that is customized to the user based on, among other things, the user's behavior, patterns, habits, preferences, and the like. The transient care plan is further influenced from the original care plan as determined by the medical entity and from the predefined health-related guidelines of the digital assistant 120. It should be noted that the transient care plan should be as similar as possible, and preferably identical, to the care plan of the medical entity (i.e., the original care plan). To that end, the desired similarity between the transient care plan and the original care plan may be predetermined as a predefined goal of the digital assistant 120.

As an example, the care plan (i.e., the original care plan) may indicate that the user should perform 25-minute sessions of physical activity four times a week. According to the same example, the dataset that has been collected with respect to the user may indicate that it would be too intense for the user to perform such physical activity based on the user's current physical fitness. According to the same example, the dataset may further indicate that the preferred physical activity of the user would be practicing yoga or walking in the park. According to the same example, the predefined health-related guidelines of the digital assistant 120 may indicate that outdoor activity shall be performed only when the temperature is within a predetermined range, that the user should drink 6-8 glasses of water per day, and so on. According to the same example, by analyzing the abovementioned example information, transient care plans that would suggest the user to go out for a walk twice a week and practice yoga once a week, may be determined and generated.

In an embodiment, the controller 200, when executing the digital assistant 120, is configured to generate the transient care plan based on the result of the analysis. It should be noted that the transient care plan may be associated with various aspects of the user's well-being, such as, the user's mental health, physical health, chronic diseases, social activity, social relationships, and so on. In an embodiment, the transient care plan may be changed after a certain duration of time when, for example, the user gets new instructions from the medical team, the medical condition of the user changes, and so on.

In an embodiment, the controller 200, when executing the digital assistant 120, is further configured to generate at least one estimated schedule for executing at least a portion of the transient care plan. Generating the at least one estimated schedule may be achieved by analyzing the transient care plan with the at least a portion of the dataset of the user. The estimated schedule includes a plurality of rules such as, but not limited to an estimated timing, for executing (or projecting) a portion of the transient care plan to a user of the digital assistant 120 via an I/O device 170. As an example, the estimated schedule may include a reminder set to 10 am for the user to take a certain medication, a suggestion set to 4 pm to go out for practicing yoga at the park with some friends, and so on. As noted above, generating the estimated schedule may be achieved by analyzing the transient care plans with at least a dataset of the user. The analysis may include applying at least one algorithm, such as a machine learning algorithm that is adapted to determine an optimal estimated schedule for the user based on the abovementioned inputs. That is, the transient care plan and the dataset of the user may be fed into the algorithm and therefore allowing to determine and create the estimated schedule (which may also be referred to as an optimal estimated schedule).

As an example, the generated transient care plan may indicate that the user shall go outside for a walk three times a week as the user suffers from back pains and therefore more intense physical activity may be too much for the user considering the back pains. According to the same example, the dataset of the user (that may be previously collected with respect to the user) may indicate that the user usually sleeps between 2-4 pm and listens to music between 4:30-5:00 μm. Therefore, the estimated schedule for executing at least a portion of the transient care plan (e.g., suggesting going out for a walk three times a week) may include a reminder that is set to 5:00 pm on Mondays, Wednesdays, and Fridays, suggesting the user to go out for a walk. It should be noted that the controller 200 may be configured to generate a daily estimated schedule, weekly estimated schedule, monthly estimated schedule, and so on. It should also be noted that to determine a daily estimated schedule, an estimated schedule for at least a week should be determined.

According to another embodiment, generating the at least one estimated schedule may include analyzing an electronic fulfillment report (or an achievement status report) indicating a fulfillment level of each of a plurality of predefined goals of the digital assistant 120. A predefined goal may include for example, nurturing the relationship (of the digital assistant 120) with the user. The fulfillment level may be for example, a score from “1” to “5”, where “1” is the lowest score indicating that the fulfillment level of a certain goal is very low, and “5” is the highest score indicating that the specific goal has been achieved. According to the same embodiment, the controller 200 may be configured to integrate at least one second plan in the at least one estimated schedule based on at least the fulfillment level of each of the plurality of predefined goals. The at least one second plan may be associated with at least one of the plurality of predefined goals of the digital assistant 120. That is, in case a certain predefined goal has a relatively low fulfillment level score, the controller 200 may integrate a respective plan that, when executed, would improve the relatively low fulfillment level score of the certain predefined goal.

In one embodiment both the at least one first plan (that is associated with the transient care plan) and the at least one second plan (that is associated with the predefined goals of the digital assistant 120) may be included in the at least one estimated schedule. According to further embodiment, the at least one estimated schedule may include only second plans that are designed to improve the fulfillment level of the predefined goals of the digital assistant 120. That is, the controller 200 may determine, based on a current state of the user, that the estimated schedule should include only plans that are designed to improve the predefined goals of the user (i.e., second plans). Thus, no plans that are associated with the transient care plan (i.e., first plans) would be integrated in the estimated schedule.

In an embodiment, the controller 200, when executing the digital assistant 120, may cause projection of at least one first plan that is associated with the transient care plan based on the at least one estimated schedule. In an embodiment, the at least one first plan may be of the at least one transient care plan that was determined. The first plan may be for example, but not limited to, a reminder to take a certain medication, a suggestion to go out for a 20-minute walk, a suggestion to start practicing yoga, a reminder to drink water, and so on. The transient care plan may include multiple plans. The plans may be projected using one or more resources of the I/O device (e.g., the resources 150 of I/O device 170, FIG. 1) that are communicatively connected to and controlled by the digital assistant (e.g., the digital assistant 120). That is, upon generation of a transient care plan and an estimated schedule for projecting at least a portion of the transient care plan, the controller 200 may be configured to project one or more first plans based on the estimated schedule. As an example, the estimated schedule indicates that a suggestion to practicing yoga should be presented to the user at 5 μm, and so when the time is 5 pm, the controller 200 executes the plan that includes a suggestion to the user to start practicing yoga.

In one embodiment, the controller 200, when executing the digital assistant, may be configured to constantly collect real-time data that is associated with a specific user and an environment in a predetermined proximity to the user using the one or more sensors (e.g., the sensors 140), and may indicate, for example, the user's mood, the specific location of the user, whether the user is awake or asleep, and more. In an embodiment, the controller 200 is configured to analyze the real-time data. The analysis may be achieved using at least one algorithm, such as a machine learning algorithm, that may be stored in a memory (e.g., the memory 220, FIG. 2). The algorithm may facilitate determination of at least a current state of at least the user based on the collected real-time data. In a further embodiment, the algorithm may be utilized to determine a current state of the environment near the user (e.g., in a predetermined proximity to the user) based on the real-time data. The current state may reflect the condition of the user and the condition of the environment near the user in real-time, or near real-time. The current state may indicate whether, for example, the user is sleeping, reading, stressed, angry, has company or not, and so on. The current state may further indicate the current time, weather, number of people in the room, people identity, and so on.

In a further embodiment, the controller 200, when executing the digital assistant 120, may be configured to identify the at least one first plan based on the estimated schedule and also on the determined current state of the user. That is, the determined current state may be utilized to update (or change) the rules of the estimated schedule for executing the at least one first plan via an I/O device. As an example, although the estimated schedule determines that a reminder to take a certain medication should be presented to the user within one minute, the current state indicates that the user has company at home so the projection of the reminder may be postponed. As another example, the user may not be near the I/O device (e.g., I/O device 170, FIG. 1) or not at home, and thus, the scheduled transient care plan may be postponed.

In an embodiment, the controller 200 may be configured to present at least a portion of the at least one estimated schedule to the user. Presenting the at least a portion of the estimated schedule to the user may be achieved using one or more resources (e.g., the resources 150) that are communicatively connected to and controlled by the digital assistant (e.g., the digital assistant 120). The estimated schedule may be presented to the user in order to, for example, increase the compliance to the transient care plan, reduce ambiguity with regard to the expected future plans, and more.

In a further embodiment, the controller 200 may be configured to collect a feedback data from the user with respect to at least one of the presented estimated schedules. The feedback data may include real-time data that may be collected using one or more sensors (e.g., the sensors 140). The feedback data refers to the user reaction to the presented estimated schedule. For example, the user may reject or accept the presented estimated schedule or a portion thereof.

In another embodiment, the controller 200 may be configured to collect feedback data from the user with respect to the abovementioned projected at least one first plan. That is, feedback data may be collected of the user in response to the already projected first plan from the I/O device 170. The user response includes the user's reaction to the plan presented via an I/O device 170. In an example embodiment, the feedback data may include, but not limited, silence, “yes” or “no” answer, free form answer, sound, movement, and more. In an embodiment, the feedback data is captured at the I/O device 170 using at least one or more sensors (140, FIG. 1) and include, without limitation, images, video, audio signals, and the like.

In an embodiment, the controller 200 is also configured to adjust and update the at least one estimated schedule based on the collected feedback data. Adjusting the estimated schedule may include for example, postpone the time at which a certain plan (e.g., a suggestion) will be executed (e.g., presented to the user), remove a certain plan from the estimated schedule, and so on. In an embodiment, the influence of the feedback data may cause a short-term adjustment of the estimated schedule (e.g., of the next action that should take place) as discussed above. In another embodiment, the influence of the feedback data may cause a long-term adjustment on the estimated schedule for the user of the digital assistant 120. As an example, for a long-term adjustment, based on the user feedback (i.e., the feedback data), the controller 200 may determine that in general, no plan (e.g., suggestion) should be presented to the user before 10 am.

In one example embodiment, when updating, for example, rescheduling, the at least one estimated schedule is required, the controller 200 may perform an automatic adjustment of the at least one estimated schedule (i.e., automatic rescheduling) without communicating it to the user. In another example embodiment, the controller 200 may suggest the user several time slots for rescheduling, such that the user would be able to choose the one she or he prefers. In yet, another example embodiment, the controller 200 may be configured to generate a question in order to ask the user for preferred time slots for rescheduling the estimated schedule. Then, based on the user preferred time slots, the controller 200 may be configured to validate that the user's preferred time slot does not collide with other events in the estimated schedule.

In an embodiment, the controller 200 is further configured to adjust the at least one estimated schedule based on a compliance level of the user to the at least one estimated schedule. The compliance level of the user to the estimated schedule indicates how many times the user accepted or rejected the suggested or executed plans when the plans were presented based on the estimated schedule. To that end, the controller 200 may be configured to collect real-time data of the user and monitor the user's behavior in real-time when, for example, plans are presented to the user. The real-time data may be collected using one or more sensors (e.g., the sensors 140, FIG. 1). Thereafter, the real-time data may be analyzed to determine the compliance level of the user to the at least one estimated schedule. As an example, when the estimated schedule includes suggesting the user to practice yoga every Monday, Wednesday, and Friday at 6 am and the user rejected each of the projected first plans, the at least one estimated schedule may be adjusted in order to improve the compliance level of the user.

In a further embodiment, although an estimated schedule for executing the transient care plan may be generated, when the user communicates with the digital assistant 120, the controller 200 may be configured to utilize the situation by adjusting the estimated schedule for executing the transient care plan in order to increase the compliance to the transient care plan. As an example, the estimated schedule indicates that at 10 am the user should start practicing yoga however, at 9:36 am the user asks the digital assistant 120 a question about the weather outside and so, the digital assistant 120 immediately adjusts the estimated schedule and therefore execute a suggestion for the user to start practicing yoga immediately.

In an embodiment, the controller 200 may be configured to determine whether there is a first gap between the care plan and the transient care plan. The first gap is a difference in at least one aspect between the care plan (i.e., the original care plan) and the transient care plan. The first gap may indicate that the instructions that were received from the medical entity (e.g., doctor), and the transient care plans that were determined by the digital assistant 120 are not synchronized. In an embodiment, the controller 200 may be configured to indicate the specific difference between the care plan and the transient care plan. As an example, while the care plan indicates that the user is required to perform physical activity three times a week, the generated transient care plan may include asking the user to perform physical activity only twice a week. In one embodiment, the transient care plan may be adjusted based on the determined first gap.

In another embodiment, the controller 200 may be configured to generate a first electronic report based on the determined first gap. The first electronic report may include a description of the first gap and the reasons for the first gap. Then, the first electronic report may be sent over a network (e.g., the network 110) to a computing device (e.g., the computing device 180) that is associated with the medical entity. By sending the first electronic report to the medical entity, the medical entity may receive information about the health-related instructions (i.e., transient care plan) the digital assistant 120 generated for the user to follow and perform.

In an embodiment, the controller 200 may be configured to determine whether there is a second gap between the transient care plan and the at least one first plan that was eventually executed by the digital assistant 120. The second gap is a difference in at least one aspect between the transient care plan and the at least one first plan. Such determination may be achieved by collecting and analyzing real-time data indicating whether the user performed according to the projected first plan (e.g., suggested) or not, and compare the analyzed real-time data to the transient care plan. Thus, in case there is a gap between the planned transient care plan and the plans that were eventually accepted and performed by the user, such a gap (i.e., the second gap) may be detected.

According to one embodiment, the transient care plan may be adjusted based on the second gap. According to another embodiment, the controller 200 may be configured to generate a second electronic report based on the determined second gap. The second electronic report may include a description of the second gap and the reasons for the second gap. Then, the second electronic report may be sent over a network (e.g., the network 110) to a computing device (e.g., the computing device 180) that is associated with the medical entity. By sending the second electronic report to the medical entity, the medical entity receives information about the plans that were performed by the user compared to the determined transient care plans.

In an embodiment, the controller 200 may be configured to determine whether there is a third gap between the transient care plan and the estimated schedule. The third gap is a difference in at least one aspect between the transient care plan and the estimated schedule. For example, due to certain constraints, some portions of the transient care plan would not be included in the estimated schedule. In such case, the controller 200 may be configured to execute a resolution process. The resolution process may include for example, adjusting the at least one estimated schedule, adjusting the transient care plan, and the like. In further embodiment, the controller 200 may be configured to generate a third electronic report based on the determined third gap. The third electronic report may include a description of the third gap and the reasons for the third gap. Then, the third electronic report may be sent over a network (e.g., the network 110) to a computing device (e.g., the computing device 180) that is associated with the medical entity.

In an example embodiment, the transient care plan may only include plans that have relatively high importance level (such as timely take medications, do physical activity, and so on) and not include plans with relatively low importance levels. And thus, plans with a relatively low importance level may not be included within the estimated schedule. However, when the user communicates with the digital assistant 120, the controller 200 may be configured to project transient care plans (a reminder, a suggestion, and the like) having a relatively low importance level such as, for example, drink water, go to sleep early, and so on.

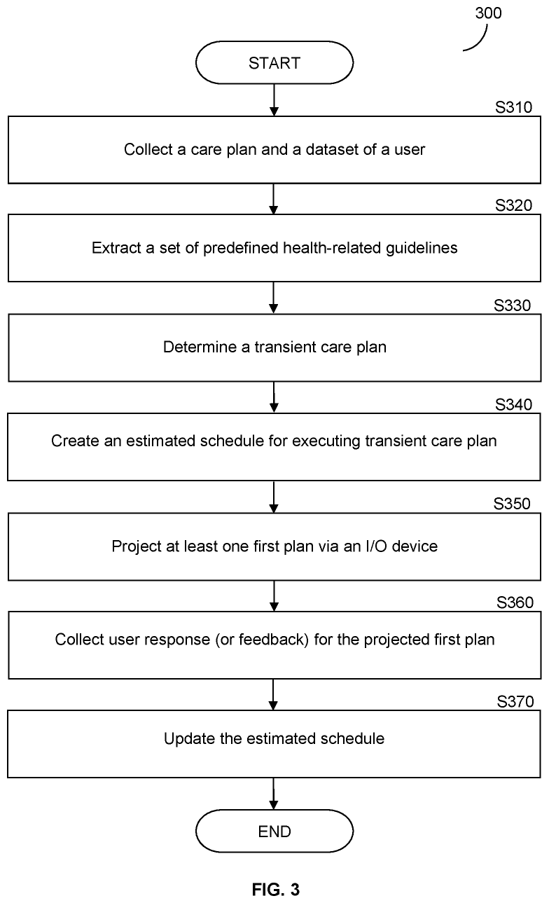

FIG. 3 shows an example flowchart 300 of a method for determining transient care plans and an estimated schedule for execution of plan for a user of the digital assistant according to an embodiment. The method described herein may be executed by the controller 200 that is further described herein above with respect to FIG. 2.

At S310, a care plan and a dataset of a user is collected. The care plan related to the user is received from a computing device that is associated with a medical entity (e.g., a doctor, medical team). The care plan may include medical instructions such as, take a certain medication at a specific timing, do physical activity three times a week, avoid red meat, and so on. The dataset of the user of the digital assistant may include at least historical data of the user (such as, preferences, behavioral patterns, etc.). In an embodiment, the dataset may also include real-time data that is being collected in real-time with respect to the user and the user's environment. The dataset of the user may be collected using sensors (e.g., the sensors 140) that are communicatively connected to and controlled by the digital assistant 120.

At S320, a set of predefined health-related guidelines of the digital assistant 120 may be extracted. The set of predefined health-related guidelines of the digital assistant 120 may refer to predetermined rules. Such health-related guidelines may indicate, for example that, the user should drink 6-8 glasses of water per day, the user should sleep at least 7 hours at night, and so on.

At S330, a transient care plan is determined based on the care plan, dataset, and the set of predefined health-related guidelines. In an embodiment, a transient care plan may be determined for each of the respective users from which the care plan and dataset are related to. In an embodiment, at least one algorithm, such as a machine learning algorithm that is adapted to determine a transient care plan, may be applied to the care plan, dataset of the user, and the set of predefined health-related guidelines. In an embodiment, the transient care plan may include a plurality of plans (e.g., suggestions, reminders, and the more) that may be applicable for the respective user. In an embodiment, the transient care plan may be associated with various aspects of the user's well-being such as, the user's mental health, physical health, chronic diseases, social activity, social relationships, and so on.

At S340, an estimated schedule for executing at least a portion of the transient care plan is created. The estimated schedule includes rules, for example, an estimated timing, for projecting each portion of the transient care plan to a user of the digital assistant. In an embodiment, the estimated schedule may be generated by analyzing the transient care plan with the at least a dataset of the user. It should be noted that other elements such as predefined goals and other limitations may have influence on the determination of the at least one estimated schedule, as further discussed above with respect to FIG. 2.

At S350, at least one first plan is projected via an I/O device. The at least one first plan is associated with transient care plan and identified based on the generated estimated schedule. The first plan may be, for example, a reminder to take a certain medication, a suggestion to go out for a 20 minutes' walk, a suggestion to start practicing yoga, a reminder to drink water, and so on. In an embodiment, real-time data about the user and the user environment may be utilized to identify the at least one first plan for projection. In an embodiment, the at least one first plan may be presented through at least one of the resources (e.g., resource 150, FIG. 1). As noted above, personalizing the transient care plan and the estimated schedule may result in the digital assistant communicating different care plans at unique times catered for different users.

At S360, a user response (of first feedback data) is collected from the user with respect to the projected at least one first plan. The first feedback data refers to the user reaction to the executed plan. As an example, the user may reject or accept the executed plan. In an embodiment, the first feedback data may include real-time data that may be collected using one or more sensors (e.g., the sensors 140, FIG. 1) and include, without limitation, images, videos, audio signals, and the like.

At S370, the estimated schedule created is updated based on the analysis of the collected user response (first feedback data). In an embodiment, analysis of the first feedback data may be achieved using one or more computer vision techniques, audio signal processing techniques, machine learning techniques, and the like. In an example embodiment, the update of the estimated schedule may include for example, postpone the time at which a certain plan (e.g., a suggestion) will be executed (e.g., presented to the user), remove a certain plan from the estimated schedule, and so on.

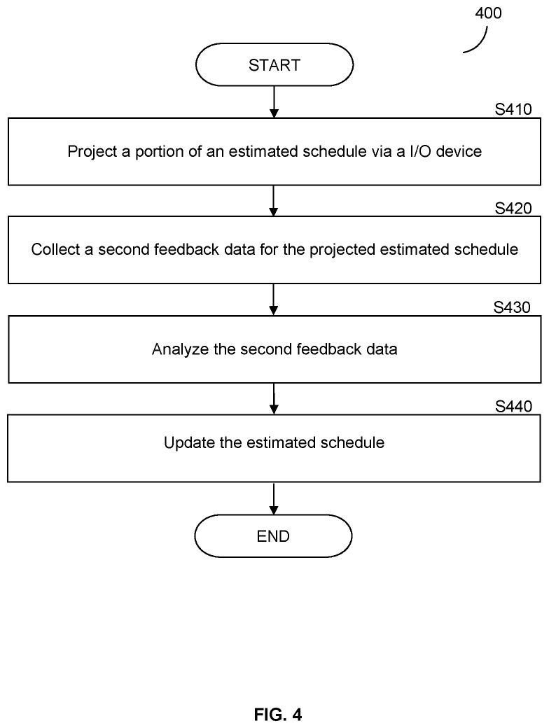

FIG. 4 shows an example flowchart 400 of a method for updating an estimated schedule for a user of the digital assistant according to an embodiment. The method described herein may be executed by the controller 200 that is further described herein above with respect to FIG. 2.

At S410, at least a portion of an estimated schedule that was previously created is presented to a user via an I/O device. The estimated schedule is utilized for executing at least a portion of the determined transient care plan at a customized schedule for each of the users of the digital assistant (e.g., the digital assistant 120, FIG. 1)

At S420, a second feedback data is collected from the user with respect to the presented estimated schedule. The second feedback data refers to the reaction of the user with respect to the presented estimated schedule. As an example, the user may reject or accept the estimated schedule. In an embodiment, the second feedback data may include real-time data that may be collected using one or more sensors (e.g., the sensors 140) and include, without limitation, images, videos, audio signals, and the like.

At S430, the collected second feedback data is analyzed. The analysis of the second feedback data may be achieved by applying one or more computer vision techniques, audio signal processing techniques, machine learning techniques, and the like.

At S440, the estimated schedule is updated based on the collected second feedback data. In an example embodiment, updating the estimated schedule may include, for example, postpone the time at which a certain plan (e.g., a suggestion) will be executed (e.g., presented to the user), remove a certain plan from the estimated schedule, and so on.

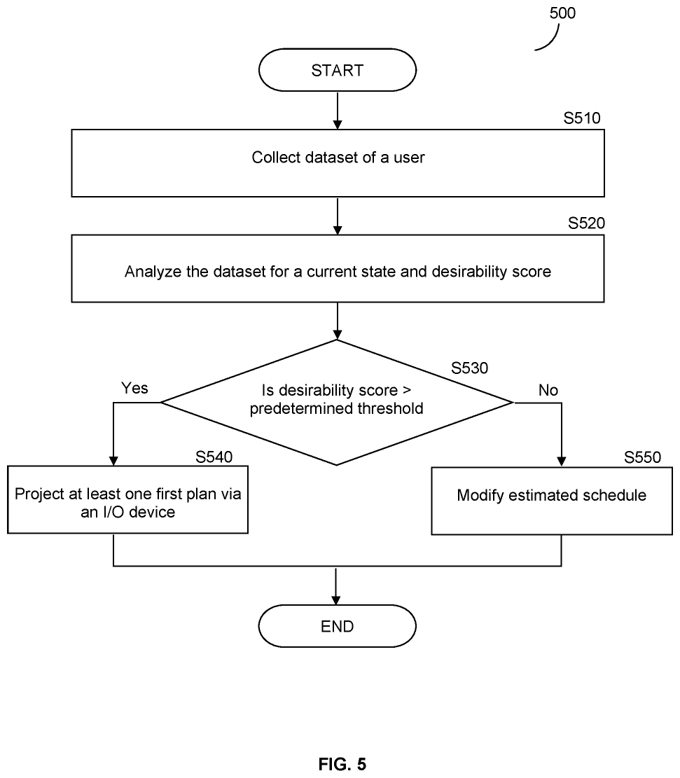

FIG. 5 shows an example flowchart 500 of a method for modifying an estimated schedule based on a current state of a user of the digital assistant according to an embodiment. The method described herein may be executed by the controller 200 that is further described herein above with respect to FIG. 2.

At S510, the dataset of a user (user dataset) is constantly or periodically collected. The user dataset of the user may include real-time data that is being collected in real-time with respect to the user and the user's environment in a predetermined proximity to the user. In an embodiment, the user dataset may also include the historical data (such as, preferences, behavioral patterns, and so on) with respect to each user of the digital assistant 120. The user dataset may be collected through time using one or more sensors (e.g., the sensors 140, FIG. 1).

At S520, collected user dataset is analyzed to determine a current state of the user and a current desirability score. The analysis may be achieved using at least one algorithm, such as a machine learning algorithm, that may be stored in a memory (e.g., the memory 220, FIG. 2). The algorithm may facilitate determination of at least a current state of the user based on at least portion of the dataset of the user. In a further embodiment, the algorithm may facilitate determination of a current state of the environment near the user (e.g., in a predetermined proximity to the user) based on at least a portion of the collected dataset of the user.

The current state may reflect the condition of the user and the condition of the environment near the user in real-time, or near real-time. The current state may indicate whether, for example, the user is sleeping, reading, stressed, angry, has company or not, and so on. The current state may further indicate the current time, weather, number of people in the room, people identity, and so on. In a further embodiment, the collected set of data may be analyzed using, for example and without limitations, one or more computer vision techniques, audio signal processing techniques, machine learning techniques, and the like.

In addition to the current state, the desirability score is determined to objectively identify the appropriate timing for projecting at least a first plan of the transient care plan. The desirability score may indicate the degree of appropriateness with respect to factors such as, but not limited to, current time, people around the user, location of user, combinations thereof, and the like.

At S530, a check is performed whether desirability score is greater than a predetermined threshold. If so, execution continues with S540; otherwise, execution continues with S550. At S540, at least one first plan is projected via an I/O device. In an embodiment, the at least one first plan of the transient care plan is executed based on a previously created estimated schedule. At S550, the estimated schedule is modified. In an embodiment, a resolution process may be performed. Such resolution process may include modifying the estimated schedule that is used for executing and projecting the transient care plan.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for executing transient care plans for a user via an input/output device, comprising:
determining at least one transient care plan based on a care plan of a user received from a medical entity, a set of predefined health-related guidelines, and at least one user dataset captured by an input/output (I/O) device, wherein a transient care plan is a customized care plan for the user;
creating, based on the at least one transient care plan and the at least one user dataset, an estimated schedule, wherein the estimated schedule includes a plurality of rules for executing a portion of the at least one transient care plan; and
projecting at least one first plan via the I/O device, wherein the at least one first plan is identified from the at least one transient care plan based on the estimated schedule.

2. The method of claim 1, further comprising:
applying a machine learning model trained to determine a current state of the user based on a real-time user data and a real-time environment data in a predetermined proximity to the user, wherein the user is a user of the I/O device; and
modifying the estimated schedule based on the determined current state.

3. The method of claim 2, wherein the real-time user data and the real-time environment data are captured by at least one sensor of the I/O device.

4. The method of claim 2, further comprising:
receiving a response by the user to the at least one first plan;
storing the received response as a first feedback data and the current state of the user as a first state of the user in a memory; and
updating the estimated schedule based on the stored first feedback data and the first state of the user.

5. The method of claim 4, further comprising:
generating a compliance level of the user based on the stored first feedback data and the first state of the user; and
updating the estimated schedule based on the generated compliance level of the user.

6. The method of claim 1, further comprising:
projecting a portion of the estimated schedule via the I/O device;
storing a response by the user to the projected portion of the estimated schedule as a second feedback data in a memory; and
updating the estimated schedule based on the stored second feedback data of the user.

7. The method of claim 1, wherein the at least one user dataset is captured by the I/O device at least one of: continuously and periodically.

8. The method of claim 1, further comprising:
determining a first gap between the care plan of the user and the at least one transient care plan, wherein the first gap is at least one difference between the care plan and the at least one transient care plan.

9. The method of claim 8, further comprising:
generating a first electronic report based on the first gap; and
providing the first electronic report to a computing device that is associated with the medical entity.

10. The method of claim 8, further comprising:
updating the at least one transient care plan based on the determined first gap.

11. The method of claim 1, wherein creating the estimated schedule further comprises:
analyzing an electronic fulfillment report including a fulfillment level for each of a plurality of predefined goals of the digital assistant, wherein the fulfillment level indicates a degree of achievement.

12. The method of claim 11, further comprising:
integrating at least one second plan in the estimated schedule based on the fulfillment level for each of the plurality of predefined goals, wherein the at least one second plan increases the fulfillment level when executed.

13. A non-transitory computer readable medium having stored thereon instructions for causing a processing circuitry to execute a process, the process comprising:
determining at least one transient care plan based on a care plan of a user received from a medical entity, a set of predefined health-related guidelines, and at least one user dataset captured by an input/output (I/O) device, wherein a transient care plan is a customized care plan for the user;
creating, based on the at least one transient care plan and the at least one user dataset, an estimated schedule, wherein the estimated schedule includes a plurality of rules for executing a portion of the at least one transient care plan; and
projecting at least one first plan via the I/O device, wherein the at least one first plan is identified from the at least one transient care plan based on the estimated schedule.

14. A system for executing transient care plans for a user via an input/output device, comprising:
a processing circuitry; and
a memory, the memory containing instructions that, when executed by the processing circuitry, configure the system to:
determine at least one transient care plan based on a care plan of a user received from a medical entity, a set of predefined health-related guidelines, and at least one user dataset captured by an input/output (I/O) device, wherein a transient care plan is a customized care plan for the user;
create, based on the at least one transient care plan and the at least one user dataset, an estimated schedule, wherein the estimated schedule includes a plurality of rules for executing a portion of the at least one transient care plan; and
project at least one first plan via the I/O device, wherein the at least one first plan is identified from the at least one transient care plan based on the estimated schedule.

15. The system of claim 14, wherein the system is further configured to:
apply a machine learning model trained to determine a current state of the user based on a real-time user data and a real-time environment data in a predetermined proximity to the user, wherein the user is a user of the I/O device; and
modify the estimated schedule based on the determined current state.

16. The system of claim 15, wherein the real-time user data and the real-time environment data are captured by at least one sensor of the I/O device.

17. The system of claim 15, wherein the system is further configured to:
receive a response by the user to the at least one first plan;
store the received response as a first feedback data and the current state of the user as a first state of the user in a memory; and
update the estimated schedule based on the stored first feedback data and the first state of the user.

18. The system of claim 17, wherein the system is further configured to:
generate a compliance level of the user based on the stored first feedback data and the first state of the user; and
update the estimated schedule based on the generated compliance level of the user.

19. The system of claim 14, wherein the system is further configured to:
project a portion of the estimated schedule via the I/O device;
store a response by the user to the projected portion of the estimated schedule as a second feedback data in a memory; and
update the estimated schedule based on the stored second feedback data of the user.

20. The system of claim 14, wherein the at least one user dataset is captured by the I/O device at least one of: continuously and periodically.

21. The system of claim 14, wherein the system is further configured to:
determine a first gap between the care plan of the user and the at least one transient care plan, wherein the first gap is at least one difference between the care plan and the at least one transient care plan.

22. The system of claim 21, wherein the system is further configured to:
generate a first electronic report based on the first gap; and
provide the first electronic report to a computing device that is associated with the medical entity.

23. The system of claim 21, wherein the system is further configured to:
update the at least one transient care plan based on the determined first gap.

24. The system of claim 14, wherein the system is further configured to:
analyze an electronic fulfillment report including a fulfillment level for each of a plurality of predefined goals of the digital assistant, wherein the fulfillment level indicates a degree of achievement.

25. The system of claim 24, wherein the system is further configured to:
integrate at least one second plan in the estimated schedule based on the fulfillment level for each of the plurality of predefined goals, wherein the at least one second plan increases the fulfillment level when executed.
